(19) United States
(12) Patent Application Publication
(10) Pub. No.: US 2017/0172502 A1
(43) Pub. Date: Jun. 22, 2017

(54) Weight Sensing System

(71) Applicant: MoMilk Ltd., Neve Ilan (IL)

(72) Inventors: Arik Rofe, Jerusalem (IL); Ofir Korech, Har Adar (IL); Michael Trakhimovich, Gan Ner (IL)

(73) Assignee: MoMilk Ltd., Neve Ilan (IL)

(21) Appl. No.: 15/118,104

(22) PCT Filed: Feb. 2, 2015
(86) PCT No.: PCT/IB15/50788

§ 371 Date: Aug. 11, 2016

Related U.S. Application Data
(60) Provisional application No. 61/938,167, filed on Feb. 11, 2014.

(51) Int. Cl. A61B 5/00; G01L 1/22

(52) U.S. Cl. CPC A61B 5/6804; A61B 5/4312; G01L 1/2243; A41C 3/04

(57) ABSTRACT

A garment is provided, configured to support a body part, an comprising a load-bearing portion configured to at least partially bear the weight of the body part, and a sensor functionally connected to the load-bearing portion so as to be acted on by the weight. The sensor comprises a core having one or more strain gauges mounted thereon, and one or more stress absorbers attached to the core. Each stress absorber 5 comprises a flexing member having a fixed end connected to the core, and a free end.

### FIELD OF THE INVENTION

The present disclosure relates to weight sensing garments, and to sensors which may be useful in such garments.

### BACKGROUND OF THE INVENTION

Many parents of infants are concerned with the amount of food their children are eating. For children who are formula fed, or whose mothers express their milk into a container for feeding, measuring their nutritional intake is a relatively simple task. For mothers of children who breastfeed, other devices and methods for measuring their intake have been devised.

In addition, may mothers who nurse find it convenient to wear brassieres specially adapted for the needs of a nursing mother. Such garments are sometimes called “nursing bras”. These garments may provide additional support which a nursing mother may need, as well as may provide convenient access for a nursing infant.

### SUMMARY OF THE INVENTION

According to one aspect of the presently disclosed subject matter, there is provided a garment configured to support a body part, the garment comprising:
a load-bearing portion configured to at least partially bear the weight of the body part; and
a sensor functionally connected to the load-bearing portion so as to be acted on by the weight;
   wherein the sensor comprises a core having one or more strain gauges mounted thereon, and one or more stress absorbers attached to the core, each stress absorber comprising a flexing member having a fixed end connected to the core, and a free end.

The garment may comprise two of the stress absorbers being connected on opposite sides of the core.

The fixed end may constitute a stopper, wherein the flexing member is designed such that the stopper is disposed within a notch formed in the core.

The flexing member may comprise an auxiliary branch terminating in an auxiliary stopper disposed within an auxiliary notch. The auxiliary notch may be formed within an extension of the flexing member.

The core may comprise sidewalls extending vertically between the stress absorbers, and upper and lower bounding members extending therebetween.

The garment bounding members may be formed with two necks, each having a smaller cross-sectional area than the rest of the bounding member on which it is formed. One of the strain gauges may be mounted on each of the necks.

The core may be formed with a channel configured to accommodate wires leading to the strain gauges.

The stress absorbers may each comprise, at an extreme end thereof, a mounting arrangement.

The mounting arrangements may comprise apertures.

The mounting arrangements may comprise one or more hooks.

The stress absorbers may be designed to isolate the core from parasitic forces.

The parasitic forces may comprise sideways-directed forces.

According to another aspect of the presently disclosed subject matter, there is provided an S-type load cell a core having one or more strain gauges mounted thereon, and one or more stress absorbers attached to the core, each stress absorber comprising a flexing member having a fixed end connected to the core, and a free end.

The load cell may comprise two of the stress absorbers being connected on opposite sides of the core.

The fixed end may constitute a stopper, wherein the flexing member is designed such that the stopper is disposed within a notch formed in the core.

The flexing member may comprise an auxiliary branch terminating in an auxiliary stopper disposed within an auxiliary notch. The auxiliary notch may be formed within an extension of the flexing member.

The core may comprise sidewalls extending vertically between the stress absorbers, and upper and lower bounding members extending therebetween.

The garment bounding members may be formed with two necks, each having a smaller cross-sectional area than the rest of the bounding member on which it is formed. One of the strain gauges may be mounted on each of the necks.

The core may be formed with a channel configured to accommodate wires leading to the strain gauges.

The stress absorbers may each comprise, at an extreme end thereof, a mounting arrangement configured to be connected to other portions of the garment. At least one of the other portions may comprise the load-bearing portion.

The mounting arrangements may comprise apertures.

The mounting arrangements may comprise one or more hooks.

The stress absorbers may be designed to isolate the core from parasitic forces.

The parasitic forces may comprise sideways-directed forces.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1 illustrates a system according to the presently disclosed subject matter;

FIG. 2 is a close-up view of a brassiere of the system illustrated in FIG. 1;

FIGS. 3A and 3B illustrate examples of placement of a sensor on the brassiere of the system illustrated in FIG. 1;

FIGS. 4 and 5 are perspective view of examples of sensors; and

FIGS. 6A and 6B are perspective views of an example of a housing for the sensors illustrated in FIGS. 4 and 5, shown, respectively, with and without a cover thereof.

### DETAILED DESCRIPTION OF EMBODIMENTS

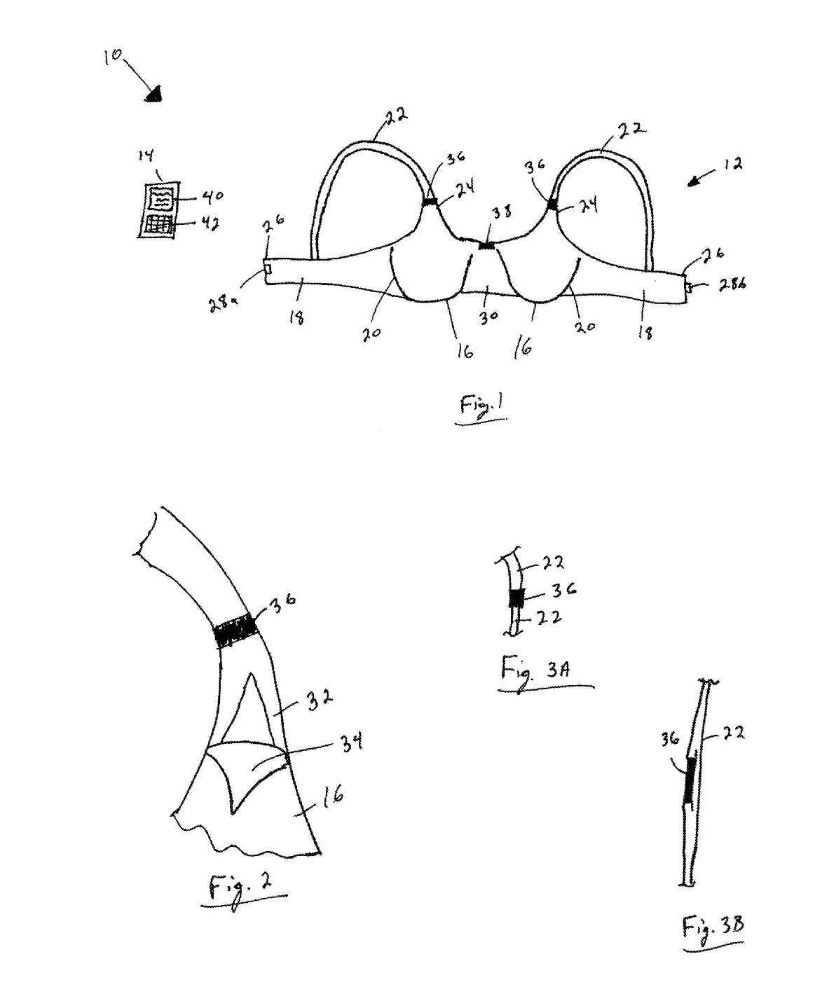

As illustrated in FIG. 1, there is provided a breast measurement system, which is generally indicated at 10. The system 10 is configured for sensing the weight of one or both breasts supported thereby, and comprises a brassiere 12 and a user interface 14. It will be appreciated that while the user interface 14 is illustrated in FIG. 1 as being external to the brassiere 12, the two may be integrated in a single unit without departing from the scope of the presently disclosed subject matter, mutatis mutandis.

The brassiere 12 may be designed to be used by a nursing woman As such, it comprises a pair of cups 16, each configured to receive therein and support a breast (not illustrated). Each cup 16 is connected to a wing 18 projecting from a side edge 20 thereof, and a strap 22 connected to an upper apex 24 thereof. Distal ends 26 of the two wings 18 are provided with cooperating fastening means 28a, 28b (collectively referred to by reference numeral 28), which may comprise hooks and eyes, a fabric hook-and-pile fastener (such as sold under the trade name VELCRO®), or any other suitable arrangement. The straps 22 extend between the apexes 24 cups 16 and the wings 18 (typically near the distal ends 26 thereof). The cups 16 are connected to one another by a bridge 30.

As illustrated in FIG. 2, each of the cups 16 may be designed to allow access to the wearer's nipples without necessitating removing the entire cup from the breast. For example, it may comprise a triangular, or other suitably shaped, frame 32, with a fabric panel 34 attached thereto so as to be at least partially removable therefrom (for example from the apex 24 thereof), for example during nursing. A securing arrangement (not shown), such as snaps or a fabric hook-and-pile fastener is provided to secure the panel 34 to the frame 32 at other times. Thus, a woman who wishes to nurse a child may partially detach the panel 34 from the frame 32, thus exposing a nipple for nursing, without the need to remove or partially remove the brassiere 12.

Reverting to FIG. 1, one or more sensors 36, configured to facilitate detecting the weight of the breasts, are provided on the brassiere 12. The sensors 36 may be or comprise, e.g., strain gauges, or any other suitable element. They are functionally connected to a load-bearing portion of the brassiere 12 so as to bear the weight of the breasts, i.e., be acted on be the weight. This may be accomplished, for example, by situating the sensors 36 within a load-bearing portion of the brassiere, for example the straps 22 thereof. According to some examples, the sensors 36 are disposed adjacent the apexes 24 of the cups 16, e.g., each sensor may connect between the apex of each cup and its associated strap 22. In addition, the brassiere 12 may comprise an accelerometer (not illustrated) at any suitable location thereon. For example, it may be provided on one of the straps 22, thereby minimizing the effect thereon by movements of the breasts.

Depending on the configuration of the user interface 14, a transmitter 38 may be provided to facilitate communication between the brassiere and the user interface. (It will be appreciated that while the transmitter 38 is illustrated in FIG. 1 as being located on the bridge 30, it may be located in any suitable location without departing from the scope of the presently disclosed subject matter, mutatis mutandis.) The transmitter 38 and the user interface 14 may configured to be connected by a wired connection, in which case each comprises suitable ports (not illustrated). Alternatively, they may be co-configured to communicate over a wireless connection, such as an audio-based channel, BLUETOOTH (i.e., one or more wireless technologies for exchanging data over short distances using short-wavelength radio transmissions in the ISM band from 2400-2480 MHz as per the standards defined by the Bluetooth Special Interest Group), NEAR FIELD COMMUNICATION (i.e., one or more technologies for smartphones and similar devices to establish radio communication with each other by touching them together or bringing them into close proximity, for example based on standards including, but not limited to, ISO/IES 18092 and those defined by the NFC Forum), WI-FI (i.e., one or more wireless local area network products that are based on the Institute of Electrical and Electonic Engineers' 802.11 standards), ZIGBEE (i.e., one or more of a suite of high level communication protocols used to create personal area networks built from small, low-power digital radios based on the Institute of Electrical and Electonic Engineers' 802.15 standard), and/or any other suitable protocols.

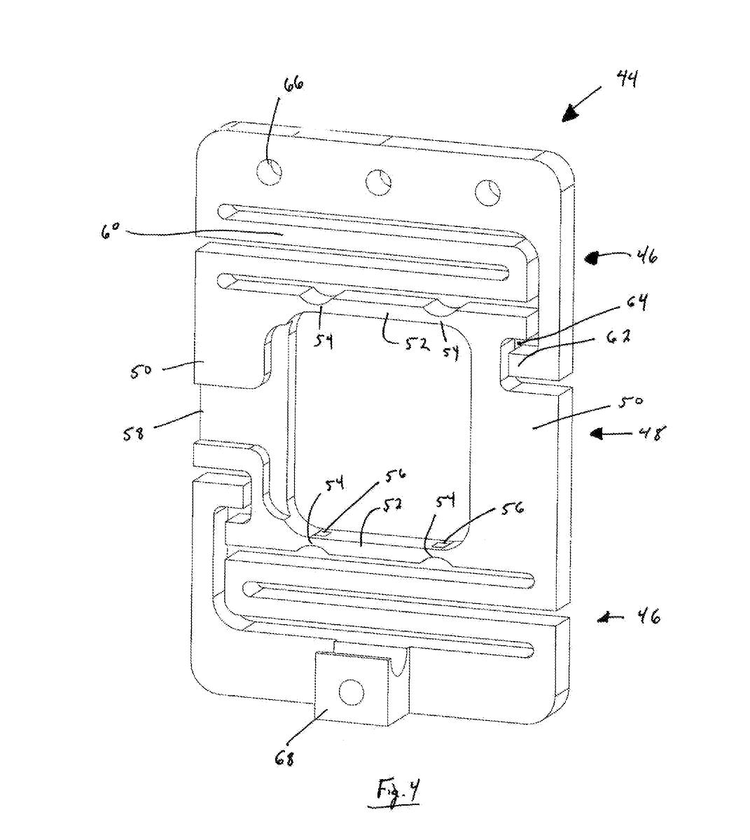

According to some examples, as illustrated in FIG. 3A, one or both straps 22 are each provided with a sensor 36 which is serially arranged with portions of the strap, i.e., portions of the strap on either side of the sensor are completely separated thereby. In this way, the sensor 36 experiences all of the stress experienced by the strap in supporting the breasts.

According to other examples, as illustrated in FIG. 3B, one or both straps 22 are each provided with a sensor 36 which is parallely arranged with portions of the strap (or other non-sensor material), i.e., portions of the strap on either side of the sensor are connected by a connecter separate from the sensor. In this way, the sensor 36 experiences, in a known way, only part of the stress experienced by the strap in supporting the breasts. According to these examples, the level of stress in each of the straps 22 can be determined based on the level of stress experienced by their respective sensors 36. This allows a sensor 36 which is more accurate at lower levels of stress to be used with more accurate results, as the amount of stress experienced thereby is reduced.

Reverting to FIG. 1, the user interface 14 is typically provided as part of a separate unit, although, as mentioned above, it may be integrated, partially or fully, into the brassiere 12. It comprises a presentation device 40 configured to present information to a user (e.g., visually, audibly, and/or a combination thereof) and one or more input devices 42, configured to facilitate a user to provide information to the system 10. According to some examples, the presentation device 40 and input device 42 are integrated in a single mechanism, such as a touchscreen (not illustrated).

The user interface 14 may be a device specifically manufactured for use with the brassiere 12. Alternatively, it may be constituted by a remote computing device, i.e., any suitable device configured to communicate with the transmitter 38, execute a program, display information to a user, and optionally receive commands from a user. It may also be configured to communicate with an external network, for example a public network such as the Internet, a POTS network, an ISDN network, cellular telephone system, and/or a VoIP system. As such, it may be any computing device, such as a mobile phone built on a mobile operating system (also referred to as a “smartphone”), a tablet computer, or any other suitable device. In particular, the remote computing device is configured for installation thereon of third-party software.

The system 10 further comprises and/or is configured to be used with a controller (not illustrated). The controller may be disposed within the brassiere 12, be external thereto, or be designed such that some elements thereof are part of the brassiere, and some are external thereto. As least some of the functionality of the controller may be performed by a remote computing device as described above. For the purposes of this disclosure, references to operations being performed by the controller may include those performed by such a remote computing device.

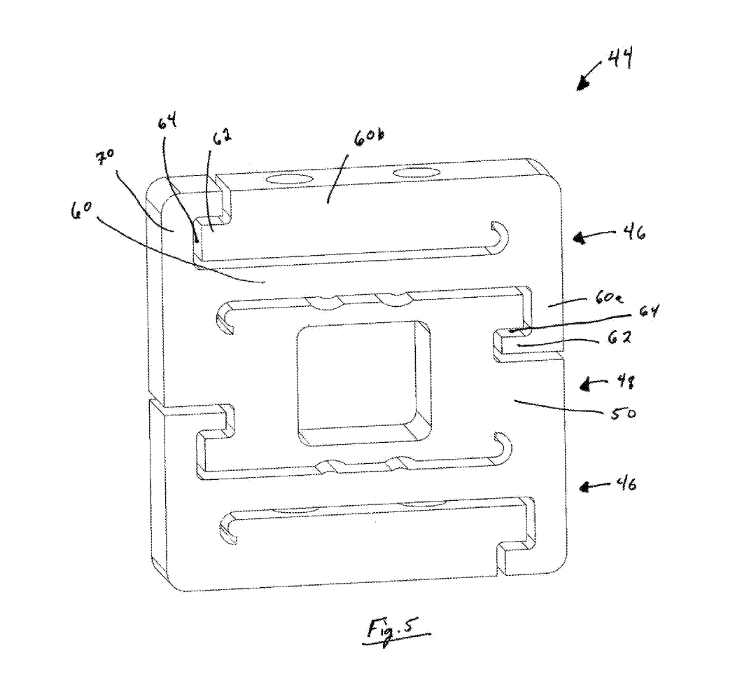

As illustrated in FIG. 4, the sensor 36 may comprise an S-type load cell 44. The load cell 44 comprises upper and lower stress absorbers 46 oppositely disposed from one another, and being connected on opposite sides of a hollow core 48. The core 48 comprises sidewalls 50, extending vertically between the stress absorbers 46, and upper and lower bounding members 52. (It will be appreciated that herein the specification and claims, directional terms such as “up”, “upper”, “down”, “lower”, “vertical”, “horizontal”, and other related terminology refer to the orientation of the sensor illustrated in FIG. 4. Additionally, directions of forces and or stresses use the same convention for describing direction.)

Each of the bounding members 52 is formed with two necks 54, each having a smaller cross-sectional area than the rest of the bounding member on which it is formed. This arrangement concentrates stresses experienced by the load cell to specific regions, i.e., those on the neck 54, facilitating measurement of the stress by measuring the strain experienced by the necks. Four strain gauges 56 (only two of which are visible in FIG. 4) are provided, one mounted on each of the necks 54. The strain gauges 56 are connected via wires or other suitable conductive elements (not illustrated) to the controller in a Wheatstone bridge configuration as is well known. Thus, the force (i.e., weight) acting on the sensor 36 can be measured. In order to accommodate the wires, a channel 58 may be formed into one of the sidewalls 50.

Each of the stress absorbers 46 is designed so as to isolate the strain gauges 56 from parasitic forces which may arise during normal use of the brassiere 12, for example due to sideway-directed motion, vibration, etc. As such, each may comprise a serpentine flexing member 60 extending between the two sidewalls 50. According to the example illustrated in FIG. 4, the flexing member comprises a fixed end connected to one of the sidewalls 50, and terminates at a free end constituting a stopper 62. The stopper is disposed within a notch 64 formed in an outer side of the other sidewall 50 (i.e., the one formed without the notch).

The flexing members 60 are configured so as to be attached, e.g., to the brassiere 12. Accordingly, the stress absorbers 46 are provided with mounting arrangements, such as apertures 66 configured to facilitate an external element to be connected thereto, and/or a hook 68 configured to facilitate an external element to be coupled therewith. Alternatively, the flexing members 60 may be provided with any other suitable mounting arrangements.

It will be appreciated that by providing the mounting arrangement on the extreme part of the stress absorbers 46 (i.e., the topmost part of upper stress absorber, and the bottommost part of the lower stress absorber), all the forces acting thereon are experienced by the entire stress absorber. This enables the load cell 44 to operate as designed, for example as described below.

As a vertical stress is applied to the load cell 44, the stress absorbers 46 deform in the direction of the stress, thereby isolating the bounding members 52, and thus the strain gauges 56, from experiencing the force. When the vertical force is great enough that the stopper 62 impacts the walls defining the notch 64, the vertical stress is experienced by the bounding members 52, and thus can be measured by the strain gauges.

A horizontal stress applied to the load cell 44, the tops of the stress absorbers 46 are pivoted in the direction of the stress. This motion is facilitated by the spaces between the flexing members 60. The movement of the stopper 62 within the notch 64 contributes to the isolation of the bounding members 52 from the stress, i.e., the stress absorbers 46 are free to pivot with minimal, if any, stress transferred to the bounding members.

Thus, the design of the load cell 44, and in particular of the stress absorbers 46 and notches 64, may facilitate isolation of the strain gauges 56 from horizontal and/or parasitic forces, as well contribute toward a small measuring resolution (e.g., on the order of a gram) of the sensor 36.

As illustrated in FIG. 5, the load cell 44 may be formed with a “double-lock” configuration, wherein the flexing member 60 comprises two branches 60a, 60b, each terminating with a stopper 62 disposed within a notch 64. One of the notches 64 may be formed within a sidewall 50, while the other may be formed within an extension 70 of the flexing member 60 provided for that purpose.

The remainder of the construction of the load cell according to the example illustrated in FIG. 5, as well as its operation, is similar to that described above with reference to the example illustrated in FIG. 4, mutatis mutandis.

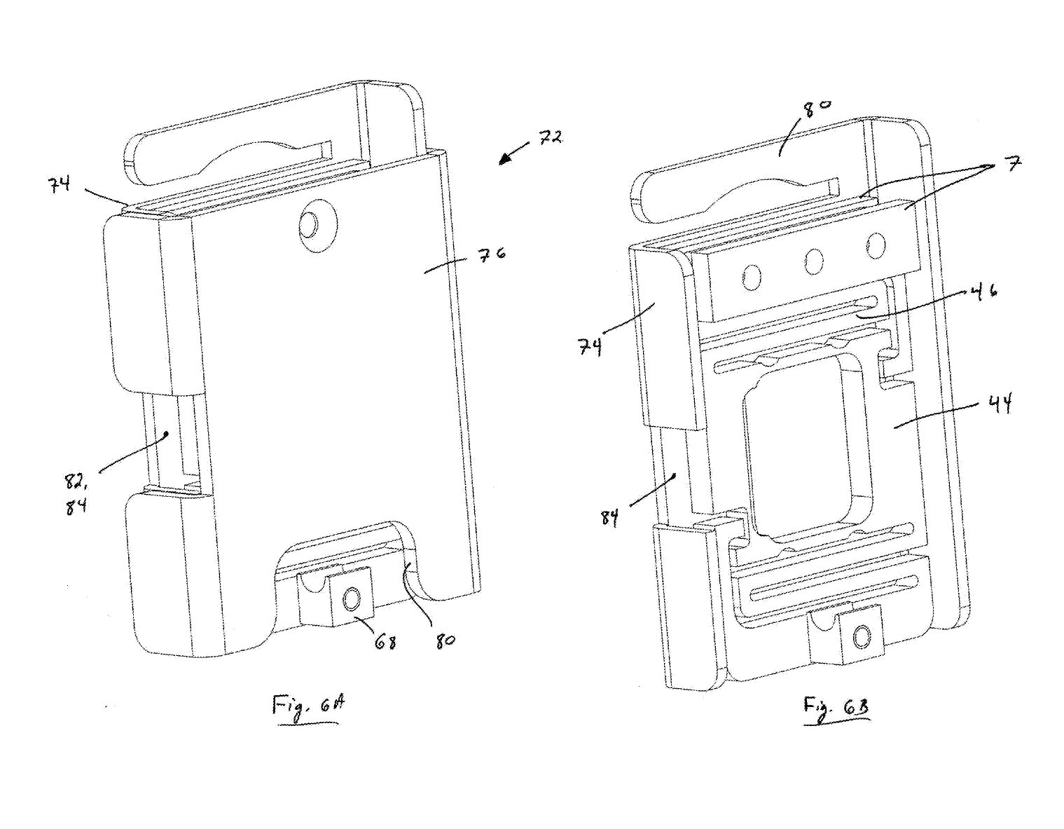

As illustrated in FIGS. 6A and 6B, a housing, which is generally indicated at 72, may be provided for the load cell 44. The housing 72 comprises a base 74 and a cover 76 (not illustrated in FIG. 6B). The base is connected to the extreme part of one of the stress absorbers 46 (e.g., the topmost part of the upper stress absorber), for example via pins (not illustrated) attached thereto and passing through the apertures 66. Spacers 78 may be provided between the load cell 44 and the housing. The base 74 is formed with a hook 80 at an upper end thereof, in order to facilitate connection to the brassiere 12, e.g., to a strap 22 thereof.

As seen in FIG. 6A, the cover 76 comprises a notch 80 on a front face thereof, providing access to the hook 68 to project therethrough, e.g., to facilitate coupling with the brassiere 12. Additional notches 82, 84 may be formed in sidewalls of the base and the housing to permit passage therethrough of the wires connected to the strain gauges 56.

As mentioned above, the system 10 may be used to measure the weight of one or more breasts supported thereby. The system 10 may be specifically configured to measure the change in weight of a breast, e.g., due to breastfeeding. Thus, it is configured to measure the weight of a breast before and after a feeding, and thereby calculate the amount of milk expressed based on the difference between the two measurements.

The system 10 may further take into account that some of the weight of the breast is not supported by the cup 16, e.g., they wings 18 and/or the wearer's body carry some of the weight, and thus are not measured by the sensors 36. A correlation between the measured weight of the breast and the actual weight of the breast can be determined experimentally. According to some investigations, every measured gram corresponds to 1.5 grams of weight. Thus, e.g., a difference of 100 grams between measurements represents a 150 difference in weight.

Alternatively or in addition, the controller may be configured to perform a calibration, wherein it calculates a correlation between the weight measured by the sensors 36 and the actual weight of the breasts. Such a calibration is based on several findings. Firstly, the factor between the measured weight and actual weight of the breast is substantially constant. Secondly, the weight of a breast can be estimated based on the size of the cup of the brassiere. Thirdly, when a user quickly rises to their toes then returns to standing normally, the breasts rise and fall accordingly, with the weight of the breasts increasing upon the descent owing to the elevated g-forces acting thereon; the additional weight during the decent is entirely supported by the brassiere.

Thus, the controller may perform a calibration, i.e., determine a factor k between the weight measured by the sensors 36 and the actual weight of the breast, such that W_{b}=kWₛ, where W_{b} is the weight of the breast, and Wₛ is the weight measured by the sensor. In order to perform the calibration, the controller instructs the wearer, for example via the presentation device 40 to quickly rise so that they are standing on their toes then return to a normal standing position (i.e., with the feet flat on the ground). The increase in Wₛ is measured by the sensors 36, and the increase in g-force acting on the breast is measured by the accelerometer. The weight of the breast under the increased g-forces is Wₛ+Δwₛ, where Δwₛ is the added weight of the breast owing to the increased g-force. As noted above, Δwₛ is substantially entirely supported by the brassiere 12. The weight of the breast under normal conditions, i.e., Wₛ, can be estimated, as noted above. Thus, the controller can compare the weights measured by the sensor 36 before and during calibration to find k. As k remains substantially constant, the controller may use this value before and after nursing to accurately determine the weight of milk expressed during a feeding.

According to another example, the controller may be configured to perform a calibration using an external element configured to apply an oscillating weight. The element may comprise a motor carrying a small eccentrically mounted load, and be configured to rotate at a predetermined angular speed. When such an element is mounted below the sensor 36 and activated, the weight of the load experienced by the sensor 36 rises and falls in a predictable fashion, based on the mass of the load and the angular speed of rotation. The controller is thus configured to correlate the voltage change of the sensor 36, and calibrate it by correlating it to the weight owing to oscillation.

Those skilled in the art to which this invention pertains will readily appreciate that numerous changes, variations and modifications can be made without departing from the scope of the invention mutatis mutandis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of.”

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A garment configured to support a body part, the garment comprising:
a load-bearing portion configured to at least partially bear the weight of said body part; and
a sensor functionally connected to said load-bearing portion so as to be acted on by said weight;
wherein said sensor comprises a core having one or more strain gauges mounted thereon, and one or more stress absorbers attached to said core, each stress absorber comprising a flexing member having a fixed end connected to said core, and a free end.

2. The garment according to claim 1, comprising two of said stress absorbers being connected on opposite sides of said core.

3. The garment according to claim 1, wherein said fixed end constitutes a stopper, said flexing member being designed such that the stopper is disposed within a notch formed in the core.

4. The garment according to claim 3, wherein said flexing member comprises an auxiliary branch terminating in an auxiliary stopper disposed within an auxiliary notch.

5. The garment according to claim 4, wherein said auxiliary notch is formed within an extension of the flexing member.

6. The garment according to claim 1, wherein said core comprises sidewalls extending vertically between said stress absorbers, and upper and lower bounding members extending therebetween.

7. The garment according to claim 6, said bounding members being formed with two necks, each having a smaller cross-sectional area than the rest of the bounding member on which it is formed.

8. The garment according to claim 7, wherein one of said strain gauges is mounted on each of said necks.

9. The garment according to claim 8, wherein said core is formed with a channel configured to accommodate wires leading to said strain gauges.

10. The garment according to claim 1, wherein said stress absorbers each comprise, at an extreme end thereof, a mounting arrangement configured to be connected to other portions of the garment.

11. The garment according to claim 10, wherein at least one of said other portions comprises said load-bearing portion.

12. The garment according to claim 10, wherein said mounting arrangements comprise apertures.

13. The garment according to claim 10, wherein said mounting arrangements comprise one or more hooks.

14. The garment according to claim 1, wherein said stress absorbers are designed to isolate said core from parasitic forces.

15. The garment according to claim 14, wherein said parasitic forces comprise sideways-directed forces.

16. An S-type load cell a core having one or more strain gauges mounted thereon, and one or more stress absorbers attached to said core, each stress absorber comprising a flexing member having a fixed end connected to said core, and a free end.

17. The load cell according to claim 16, comprising two of said stress absorbers being connected on opposite sides of said core.

18. The load cell according to claim 16, wherein said fixed end constitutes a stopper, said flexing member being designed such that the stopper is disposed within a notch formed in the core.

19. The load cell according to claim 18, wherein said flexing member comprises an auxiliary branch terminating in an auxiliary stopper disposed within an auxiliary notch.

20. The load cell according to claim 4, wherein said auxiliary notch is formed within an extension of the flexing member.

21. The load cell according to claim 16, wherein said core comprises sidewalls extending vertically between said stress absorbers, and upper and lower bounding members extending therebetween.

22. The load cell according to claim 21, said bounding members being formed with two necks, each having a smaller cross-sectional area than the rest of the bounding member on which it is formed.

23. The load cell according to claim 22, wherein one of said strain gauges is mounted on each of said necks.

24. The load cell according to claim 23, wherein said core is formed with a channel configured to accommodate wires leading to said strain gauges.

25. The load cell according to claim 16, wherein said stress absorbers each comprise, at an extreme end thereof, a mounting arrangement.

26. The load cell according to claim 25, wherein said mounting arrangements comprise apertures.

27. The load cell according to claim 25, wherein said mounting arrangements comprise one or more hooks.

28. The load cell according to claim 16, wherein said stress absorbers are designed to isolate said core from parasitic forces.

29. The load cell according to claim 28, wherein said parasitic forces comprise sideways-directed forces.
